(19) United States
(12) Patent Application Publication
(10) Pub. No.: US 2007/0272664 A1
(43) Pub. Date: Nov. 29, 2007

(54) Carbon and Metal Nanomaterial Composition and Synthesis

(76) Inventors: Kurt A. Schroder, Coupland, TX (US); Karl Matthew Martin, Austin, TX (US)

Correspondence Address: Jerry M Keys;Winstead, PO box 50784, Dallas, TX, 75201, US

(21) Appl. No.: 11/659,409

(22) PCT Filed: Aug. 4, 2005
(86) PCT No.: PCT/US05/27711

§ 371 Date: Feb. 2, 2007

(51) Int. Cl. B23H 11/00; A61K 9/00

(52) U.S. Cl. 219/121.59; 424/484; 977/775

(57) ABSTRACT

The invention relates generally to nanopowder synthesis processes, and more particularly to the controlled use of a precursor material (such as a precursor gas) to assist in the formation of unagglomerated nanoparticles of the powder. It also relates to novel nanomaterials comprised of carbon and metals produced by the process along with the fundamental processes the novel nanomaterials enable.

### FIELD OF TEE INVENTION

The invention relates generally to nanopowder synthesis processes, and more particularly to the controlled use of a precursor material (such as a precursor gas) to assist in the formation of unagglomerated nanoparticles of the powder. It also relates to novel nanomaterials comprised of carbon and metals produced by the process along with the fundamental processes the novel nanomaterials enable.

### BACKGROUND

In the area of material powders, metal is used in many applications including electrically and thermally conductive pastes, photographic films, antibacterial agents and conductive inks. Most of the current applications use micron or sub micron powders. Recently, several processes have demonstrated commercial scale of nanopowders, some including metals. Nanopowders exhibit unique properties that are different than their micron counter-parts such as lower melting/sintering temperatures, higher hardness, increased optical transparency and increased reactivity. Many applications would like to benefit by exploiting these properties. Until recently, the commercial availability of nanopowders has been limited to a few materials such as silica, carbon black and alumina. Several new processes are now producing nanopowders in commercial scale and have the ability to make a wide range of materials including silver, copper, gold, platinum, titanium and iron as well as others.

An important aspect of these powders is that the particles generally need to be unagglomerated. This aids in preserving the properties unique to the nanoscale and allows easier incorporation of the powder into most applications. Many of the new processes, especially with metals, cannot produce unagglomerated particles. Metal particles at this size have high surface energies and are consequently unstable. When two particles contact one another, the particles form a neck to decrease both the local curvature and surface area, consequently lowering the total surface energy. The result is the formation of hard agglomerates, or aggregates, of the nanopowder which are nearly impossible to break apart. Since the particles are fused to one another, they begin to act like a much larger particle and lose many of the desired characteristics of nanoparticles. In reality, the particles form a nanostractured material instead of a true nanopowder. When this happens it is nearly impossible to process the particles down to their original primary particle size either by chemical or mechanical means. This is especially true in gas phase condensation synthesis techniques in which the particles are formed at elevated temperatures, meaning that the particles have even higher energies and are colliding with one another while at an elevated temperature. Nano-sized particles have been shown to have reduced melting and sintering temperatures relative to the bulk material properties. This makes nanopowders very prone to aggregation at elevated temperatures.

Several methods have been tried to eliminate this problem. Some processes such as Sol-Gel chemistry can produce lab scale quantities of nano-metal particles in a solution which form discrete, unagglomerated particles by incorporating specific surfactants or ligands that bond to the particle's surface to prevent the particles from contacting one another while in solution. However, when the solvent is evaporated to isolate the particles from the solution, the particles typically form aggregates. Other researchers at the University of Bologna, Italy reported dodecanethiol coating of silver nanoparticles in an aqueous solution to avoid the agglomeration. These methods have the limitation that the particles form hard agglomerates when the solution is dried to extract the powder; therefore they are limited to applications where the modified particle surface chemistry, the chemistry of the particle-solution and the chemistry of the application solutions are compatible. Additionally, these processes are not amiable to large-scale production due to the high cost and difficulties associated with scaling the batch process.

Another method uses a sodium/halide flame and encapsulation technology (SFE) to form discrete nanoparticle powders. This process uses a three-inch long flame inside a four-foot long tubular flow reaction furnace for sodium reduction of metal halides, such as boron trichloride and titanium tetrachloride, to produce metal and ceramic nanoparticles. The particles produced are 10 to 100 nm in diameter with a salt encapsulation. This system is an open loop process that requires continuous feed of the salt encapsulation solution and the combustion gases into the reaction furnace. Hence, it uses considerable gases and is not very efficient. Lastly, for most applications, this material requires an additional step to remove the salt encapsulation. The salt encapsulation can present chemical compatibility issues, especially in applications where ionic contamination is not well tolerated, even when the encapsulation is removed.

The Harima Electronic Material division of Harima Chemicals based in Tokyo, Japan uses a gas evaporation process to produce a nano-silver paste containing particles with an average size of 7 nm coated with an organic dispersing agent. This material has much of the same issues as Sol-Gel produced material in that the dispersant agent that is bonded to the particle's surface must be removed from the silver to have the silver reactive. Additionally, if the paste is dried to form a powder, the particles become aggregated.

Another method described in the publication “Production of carbon-coated aluminum nanopowders in pulsed microarc discharge” published Sep. 16, 2002, in Nanotechnology 13 (2002) 638-643 describes the use of a 1-50 V and 30-150A, 200 microsecond, microarc discharge between closely spaced electrodes (0.01-0.1 mm) of aluminum and copper in a 1 atm natural gas or methane environment to produce microscopic quantities of 23 nm aluminum particles with a 1 nm carbon coating. This process contained no gas controls and is an open loop system requiring working gases and carrier gas. It produced metals with inconsistent morphologies. Generally, the particles have a metastable amorphous morphology. Amorphous morphology is generally not desirable for metal particles because the particles will crystallize over time and/or at temperature resulting in unstable reactivity of the particles. Lastly, in this process, the microscopic quantities of particles were collected 3mm from the arc by drifting onto a substrate, again further demonstrating that the technology is not commercially feasible. Another method for producing unagglomerated nano-particles is described in U.S. patent application Ser. No. 10/669,858 (“the '858 Patent Application”), which patent application is commonly owned by the Applicant of the Application and the invention disclosed therein is referred hereinafter as “the Solenoid process” or “the '858 Patent Application process.” In the Solenoid process, a pulsed solenoid is used in conjunction with a high power, pulsed plasma (500-5000+V, 10,000-100,000+A, 0.1-10 ms) process to produced unagglomerated nano-particles in commercial quantities. In this application, the liner of the solenoid provides an uncontrolled precursor for coating the particles. In operation, the plasma created from the metal precursor materials used to make the nanopowders evaporates the liner. The amount of material removed from the liner is not controlled.

Additionally, the gas species evolved by the vaporization of the liner is not controlled and is dependent upon the liner composition and production conditions. The liner is restricted to materials that are compatible with this process and limits the choice of particle coating materials to a very short list of high strength, plasma tolerant and insulating materials. Hence, it is impossible to control the coating precursor concentration within this process.

The material produced from the Solenoid process consisted of discrete metal particles surrounded by carbonaceous material. Because the silver is not tightly bound to the carbon material and there is no surface chemistry attached to the silver particles, they are very active. Specifically, 25nm silver nanoparticles were produced that have been shown to have good bacterial efficacy in a commercial topical wound dressing.

So while chemistry methods are capable of producing discrete, unagglomerated nanometals in solutions, they generally are not commercially viable and the particles contain surface chemistry that is often not compatible with formulations and can adversely affect the uniqueness of the nano-properties. Other processes can produce dry nano metal particles, however they contain surface chemistries that are undesirable, the particles are aggregated or the processes are not commercially viable. Therefore, there remains a need to produce commercial quantities of unagglomerated nanometals in dry powder form.

### SUMMARY OF THE INVENTION

The current invention overcomes the previous art problems and difficulties, by producing dry, unagglomerated coated nanopowders in commercial volumes in a controllable process. The particles are stable at room temperature and remain discrete. The new process can use a similar high-powered, pulsed plasma process as disclosed and described in U.S. Pat. No. 6,777,639 (“the '639 Patent”) and the '858 Patent Application, but without the complexity of the pulsed solenoid used in the Solenoid process. Additionally, unlike the Solenoid process, the current invention provides a high level and wide range of control of coating properties and coating precursors. The current invention produced far-reaching results and produced both non-agglomerated nanoparticles and novel nanomaterial compositions.

The invention in the broad extent provides a novel method for synthesizing nanometals as well as a method for producing novel nano-materials. In some embodiments, the synthesis process incorporates a system for automatically controlling the coating precursor material within the synthesis process. The controlled coating precursor system can be in multiple forms including a controlled gas, liquid or solid feed system or combination therein. The coating precursor may interact with the plasma, the particles or combinations therein. By using these methods of controlled coating precursor, a wide range of particle sizes and coatings can be achieved.

In an embodiment of the invention, the control of the coating precursor material is accomplished by using a gas injection control system to provide a controlled hydrocarbon precursor material that interacts with the synthesis process to produce highly unagglomerated nanometal particles. The hydrocarbon gas interacts with the plasma and nanomaterial precursor material to form carbonaceous materials that assists in keeping the nanoparticles unagglomerated. Additionally control of the agglomeration level is accomplished by control of the hydrocarbon gas species and quantity.

In another embodiment of the invention, a gas evolving system is used to introduce the hydrocarbon precursor into the system to control the amount of particle agglomeration. In this embodiment, a solid or liquid precursor is used to evolve gas in a controlled manner into the synthesis process. The gas evolution may occur by interaction with the plasma or by an independent source such as heating the solid or liquid. For instance, a solid hydrocarbon precursor rod can be fed into the process in a controlled manner to evolve the hydrocarbon gas.

In another embodiment of the invention, the hydrocarbon gas is created by controlled injection of a liquid hydrocarbon precursor into the process to interact with the plasma. The hydrocarbon gas may also be created by controlled evaporation of the liquid precursor material.

The process of the current invention produces novel materials. In some embodiments, the novel materials are a composite of unagglomerated nanometals and a carbonaceous material. The carbonaceous material has been shown to contain a carbyne form of carbon.

Furthermore, the material produced by the process has been shown to be effective against a wide range of bacteria. For instance, the silver material embodiment of the present invention has been shown to have bacterial efficacy against both gram positive and gram-negative bacteria.

### LIST OF DRAWINGS

FIG. 1 is a diagram of the pulsed power synthesis system embodiment of the present invention that is configured with an automated gas control system for the coating precursor material.

FIG. 2 is a TEM image of 77 nm silver produced without any coating precursor.

FIG. 3 is a TEM image of a composition embodiment of the present invention (45 nm silver produced using 44 ppm of acetylene gas).

FIG. 4 is a TEM image of another composition embodiment of the present invention (28 nm silver produced using 440 ppm of acetylene gas).

FIG. 5 is a TEM image of another composition embodiment of the present invention (22 nm silver produced using 4,400 ppm of acetylene gas).

FIG. 6 is a TEM image of another composition embodiment of the present invention (9 nm silver produced using 44,000 ppm of acetylene gas).

FIG. 7 is a TEM image of another composition embodiment of the present invention (30 nm silver produced using 8800 ppm of methane gas).

FIG. 8 are the XRD plots of 25 nm material produced by the Solenoid process.

FIG. 9 are the XRD plots of another composition embodiment of the present invention (25 nm material produced using 4400 ppm acetylene).

FIG. 10 are the XRD plots of another composition embodiment of the present invention (10 nm material produced using 44,000 ppm acetylene).

FIG. 11 are the XRD plots of the composition embodiment of the present invention of FIG. 7 (the 30 nm material produced using 8800 ppm methane).

FIG. 12 is a TEM image of the composition embodiment of the present invention of FIG. 9 (25 nm silver produced using 4,400 ppm acetylene), which shows the carbyne structures.

FIG. 13 is the EELS data of the composition embodiment of the present invention of FIGS. 9 and 12 (25 nm silver produced using 4,400 ppm acetylene), which confirms the presence of carbynes and discrete silver particles.

FIG. 14 is a TEM image of the composition embodiment of the present invention of FIG. 10 (10 nm silver produced using 44,000 ppm acetylene), which shows the carbyne structures and discrete silver particles.

FIG. 15 is a TEM image of the carbyne structures of the composition embodiment of the present invention of FIGS. 10 and 14 (10 nm silver produced using 44,000 ppm acetylene), which shows the presence of carbynes.

FIG. 16 is a TEM image of a prior art carbon/silver composite.

FIG. 17 is another TEM image of the same prior art carbon/silver composite of FIG. 16.

FIG. 18 is another TEM image of the same prior art carbon/silver composite of FIGS. 16 and 17, which shows crystalline particles.

FIG. 19 is another TEM image of the same prior art carbon/silver composite of FIGS. 16-18, which shows crystalline particles.

FIG. 20 is a TEM image of the same prior art carbon/silver composite material of FIGS. 16-19, which shows only the carbon material.

FIG. 21 is a TEM image of the prior art carbon/silver composite material of FIGS. 16-20, which shows only the carbon material.

FIGS. 22A-C are TEM images of a composition embodiment of the present invention (silver/carbon composite material made using 8800 ppm methane), which shows the presence of carbyne.

FIG. 23 shows EELS analysis of the composition embodiment of the present invention of FIGS. 22A-C (silver/carbon composite material made using 8800 ppm methane), which confirms the presence of carbyne.

FIGS. 24A-D are TEM images of another composition embodiment of the present invention (copper/carbon composite produced using 44,000 ppm acetylene), which shows the presence graphitic and fullerene carbon.

FIG. 25 is the EELS data of the composition embodiment of the present invention of FIGS. 24A-D (copper/carbon composite produced using 44,000 ppm acetylene), which confirms the presence graphitic and fullerene carbon.

FIGS. 26A-B are TEM images of another composition embodiment of the present invention (iron/carbon composite produced using 4,400 ppm acetylene), which shows the presence graphitic and fullerene carbon.

FIG. 27 is the EELS data of the composition embodiment of the present invention of FIGS. 26A-B (iron/carbon composite produced using 4,400 ppm acetylene), which confirms the presence graphitic and fullerene carbon.

FIGS. 28A-B are TEM images of another composition embodiment of the present invention (iron/silver/carbon composite/alloy material using acetylene).

FIGS. 29A-B are TEM images of another composition embodiment of the present invention (carbon material using carbon precursor material and acetylene gas).

FIG. 30 is a diagram of another pulsed power synthesis system of the present invention that is configured with an automated liquid control system for the coating precursor material.

FIGS. 31A-B are TEM images of another composition embodiment of the present invention (silver/carbon composite material using 10 gm heptaethiol).

FIG. 32 is a TEM image of another composition embodiment of the present invention (silver/carbon composite material using 20 gm heptaethiol).

### DETAILED DESCRIPTION OF PREFERRED EMBODIMENTS

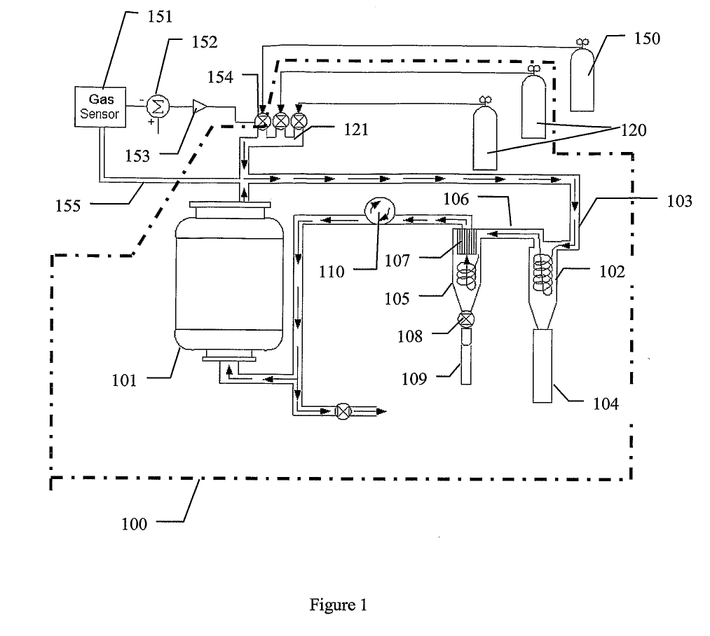

The current invention alleviates the problems of the previous systems and provides a unique system that has the ability to control the agglomeration of the particles and is versatile enough to handle different coating precursors. FIG. 1 shows a detailed schematic of the current invention. The current invention uses a gas injection system with a continuous closed loop feedback concentration control system to control the hydrocarbon precursor to assist in forming the unagglomerated nanometals.

The system is composed of the radial gun synthesis process 100 described in the '639 Patent, of which is incorporated by reference and which details have been omitted. The reaction vessel 101 is connected to the cyclone 102 via a collection pipe 103. The cyclone is used to remove larger particles, typically greater than 0.5 micron, which are collected in the cyclone hopper 104. The cyclone is connected to the dust collector 105 by a stainless steel pipe 106. Located within the dust collector is a filter 107 used to separate the powder from the gas stream. The bottom of the dust collector contains a packaging valve 108 which is connected to a packaging container 109 used to collect the powder. The outlet of the dust collector is connected to the inlet of a sealed blower 110. The outlet of the sealed blower is then connected to the reaction vessel 101 to form a closed loop system.

Additionally, gas bottles 120, typically helium and nitrogen, are connected to a gas injection manifold, 121. The new invention incorporates a bottle of particle coating precursor gas 150, such as a hydrocarbon gas like acetylene or methane, connected to the gas injection valve 154. While the preferred embodiment uses hydrocarbon gases it is not limited to hydrocarbon gases and other gases such as silane can be used. A gas sensor 151 is connected to the outlet of the reaction vessel and pulls gas samples out of the reaction vessel. The gas sensor contains a set point controller 152 that uses the data from the gas sensor to maintain a predefined gas concentration.

In operation, the system is vacuumed to remove any oxygen from the system and is then filled with the inert gases. The inert gases may be, but are not limited to argon, helium, nitrogen, and neon. The blower 110 is turned on and the gases are recirculated. The set point controller 152 is set to maintain a specific gas concentration, typically in the range of 1-500,000 ppm and more specifically in the range of 50-50,000 ppm, and then the hydrocarbon gas is injected into the system. The gas sensor 151 monitors the gas concentration and the set point controller causes the gas injection valve 154 to inject the hydrocarbon gas to maintain the specified concentration. The material synthesis is started and powder is produced. As powder is produced, the coating precursor material is consumed and additional coating precursor material is automatically added to the system. As the powder is produced, the blower 110 is continuously recirculating the gas within the closed loop. The powder moves with the gas through the cyclone 102 where larger particles are removed. The remaining powder continues to the dust collector 105 where it collects on the surface of the filter 107. The filter is back pulsed with compressed gas (not shown) to remove the powder and allow it to fall into the packaging container 109. The packaging valve 108 can be closed to seal the system and allow the packaging container with the powder to be removed. The gas that passes through the filter then flows into the blower and is sent back to the reaction vessel.

During the synthesis process, the hydrocarbon gas interacts with the plasma. As the gas quenches, it may form solids, react with the metal vapor or may catalytically interact with the metal vapor and particles. The resulting product of the plasma and gas quench is the formation of highly unagglomerated metal nanoparticles. Additionally carbon structures, including amorphous carbon (soot), graphite, fullerenes, carbon nanotubes, diamond like carbon structures and carbyne structures and combinations thereof may be formed. The carbon may interact with the inert gases to form other compounds such as cyano derivatives in the case of nitrogen. Additionally, for some metals such as aluminum, compounds may be formed that contain carbide compounds. Consequently the hydrocarbon gas is being consumed and must be adjusted to maintain a specific gas concentration.

While the current examples show materials produced using acetylene and methane, other gases such as alkanes (methane CH4, ethane C2H6, propane C3H8, butane C4H10, pentane C5H12, heptane C6H14, etc.), alkenes, alkynes at an appropriate but non-explosive vapor pressure could be used with the current invention. While the current examples use hydrocarbon gas, it is not necessarily limited to them. For example silane gas could be used to form a silicon matrix or borane gas could be used to form a boron matrix when a portion of the matrix gas does not form a compound with the metal precursor. One skilled in the art will also recognize that mixture of gases could also be used. In some cases it may be possible to form combinations of the metal particles, compounds of the metal and matrix gas and the matrix. Other gases such as organo-metallic gases such as ferrocene could also be used.

The current invention has broad capabilities and demonstrated the ability to produce a wide array of material sizes, morphologies and compositions. In producing materials, the new invention was able to produce materials in the range of 8-100 nm with precise and consistent control, far greater than the solenoid process. For comparison purposes, the solenoid process was able to produce material down to 25 nm; however, it could not produce this material consistently. The new process was also able to produce new materials that contained higher carbyne contents and compositions that were pyrophoric and had different dispersibility characteristics. Additionally, the new process is capable of producing various materials including but not limited to metals, metal alloys and combinations of metals and metal alloys. More specifically, silver, copper, aluminum, iron, nickel, combinations thereof, alloys thereof and combinations of the metals and alloys thereof were produced. One skilled in the art will recognize that other metals such as zirconium, niobium, gold, platinum, cobalt, titanium, zinc, hafnium, tantalum, tungsten, alloys thereof and mixtures thereof can be used in the process. The following shows some examples of materials produced by the current process.

### EXAMPLE 1

The following tests were performed using the system invention of the present Application. The radial gun synthesis technique as described above was used to produce the material. A commercial dead-band feedback controller, Omega CNI 3222-C24, and hydrocarbon sensor, VIG industries FID Model 20, were integrated into the gas control system. Acetylene and methane were used for the hydrocarbon gas. All size measurements are computed based on BET measurements and an equivalent sphere diameter model.

The tests were conducted using the feedback controller to maintain specific levels of hydrocarbon including 0 ppm, 44 ppm, 440 ppm, 4400 ppm and 44,000 ppm in an atmosphere otherwise composed of helium and nitrogen at a total pressure of 1 atmosphere. All the tests were performed with the same production conditions with only the hydrocarbon and make-up gas concentrations being varied. Results are shown in Table 1.

TABLE 1

| Gas Concentration | Hydrocarbon | | |
|---|---|---|---|
| (ppm) | Gas | BET (nm) | Agglomeration |
| 0 | None | 77 | Highly necked |
| 44 | Acetylene | 45 | Slightly necked |
| 440 | Acetylene | 28 | Mostly |
| | | | unagglomerated |
| 4400 | Acetylene | 22 | Unagglomerated |
| 44,000 | Acetylene | 10 | Unagglomerated |
| 8800 | Methane | 30 | Unagglomerated |

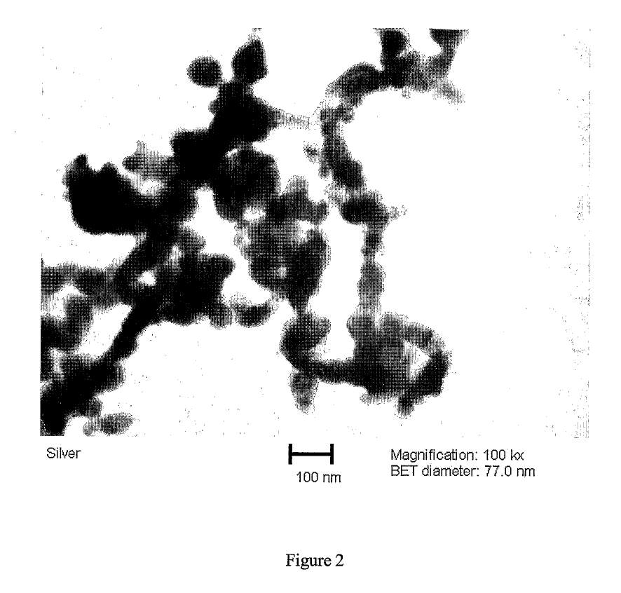

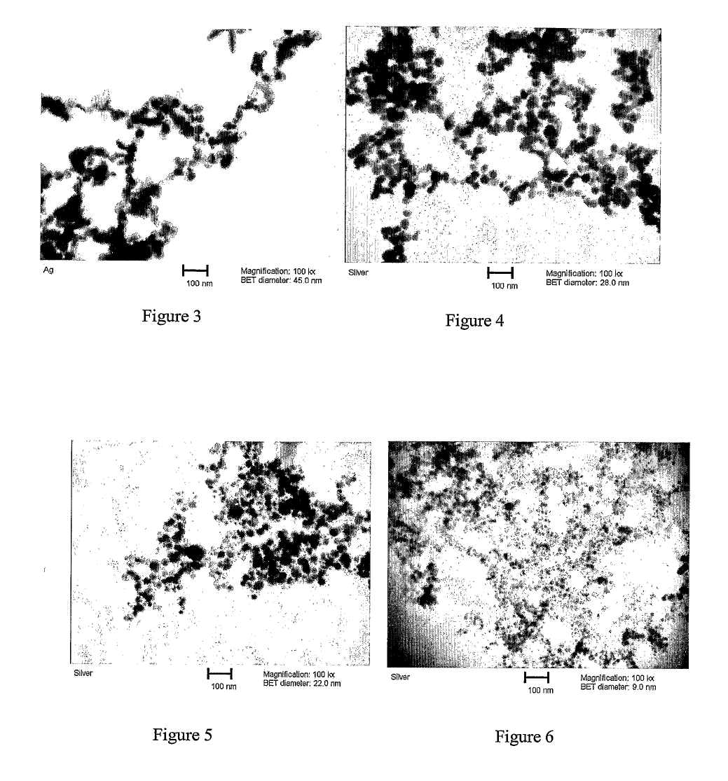

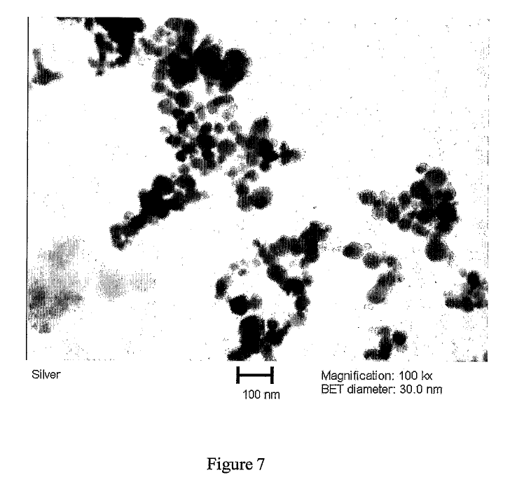

FIG. 2 shows the 77 nm silver produced with no hydrocarbon gas. It shows extensive necking between particles. FIG. 3 shows the 45 nm silver produced by adding only a 44-ppm concentration of acetylene. The amount of agglomeration is substantially reduced. FIG. 4 shows 28 nm silver produced by adding 440-ppm acetylene. FIGS. 5 and 6 show material produced at 4400 ppm and 44,000 ppm levels of acetylene. The material size produced was 22 nm and 9 nm, respectively. This shows a clear trend of decreasing particle size with increasing gas concentration. Additionally the particles are discrete and unagglomerated.

Even smaller silver, such as having a material size as low as 8 nm, was achieved by farther increasing the acetylene concentration. Thus, embodiments of the invention have an average size of material in the range between about 8 n, and about 45 nm, and more specifically, can have a size of material in the range between about 8 nm and about 25 nm, and even more specifically, can have a size of material in the range between about 8 nm and 15 nm. Additional tests were performed using methane at 8800-ppm concentration using the same production conditions as above. The results of the material produced are shown in FIG. 7. This material is also unagglomerated.

The material from the current process has been analyzed to determine its uniqueness. TEM, Static light scattering (SLS) and dynamic light scattering (DLS) have been used to determine the extent of agglomeration and to quantify the dispersibility of the material. Inductively coupled plasma Optical Emission Spectroscopy (ICP), X-ray diffraction (XRD), LECO and Electron Energy Loss Spectrometry (EELS) were used to determine the material composition and morphology. Fourier Transform Infrared (FTIR) and gas chromatography/mass spectroscopy (GC/MS) were used to identify hydrocarbons.

The Table 2 summarizes some of the powder characteristics as determined by various tests. The mean particle size is computed from an equivalent sphere diameter model based on the surface area measurements from the Monosorb BET. The Brunauer, Emmett and Teller (BET) method for particle measurement uses gas adsorption to determine the specific surface area that is then used to compute a particle size based on an equivalent sphere model. The mean particle size in various liquids was determined by a Horiba LA910 SLS. In each SLS test, a 20 gm, 0.1% solution was prepared in a beaker and sonicated using a Misonix Sonicator 3000 with a 0.5-in probe for 2 minutes (4 minute elapsed) at 90% power, half duty cycle. The carbon content of each powder was measured based on LECO analysis.

TABLE 2

| | BET | | Carbon | SLS D50 (nm) | | |
|---|---|---|---|---|---|---|
| | (nm) | Gas Conc. (ppm) | Mass % | Water | IPA | Ethanol |
| Solenoid | 25 | — | 3 | 328 | 257 | 148 |
| Acetylene | 25 | 4,400 | 3 | 155 | 205 | 243 |
| Acetylene | 10 | 44,000 | 30 | 392 | 900 | 2185 |
| Methane | 30 | 8,800 | 1.5 | 215 | 220 | 171 |

From these results, the material from the new process is distinguishable from the solenoid process. Additionally, for a given gas the carbon content increases with the amount of gas in the reaction chamber. Interestingly, for a given ambient precursor gas carbon content (4,400 ppm acetylene vs. 8,800 ppm methane) the carbon content in the materials is not the same. This indicates that the coating composition is different for the different gases.

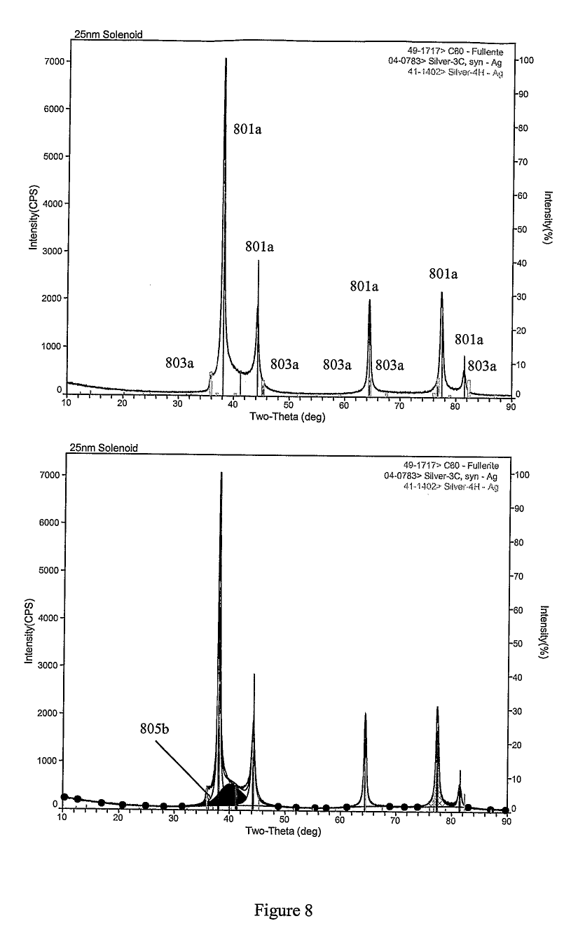

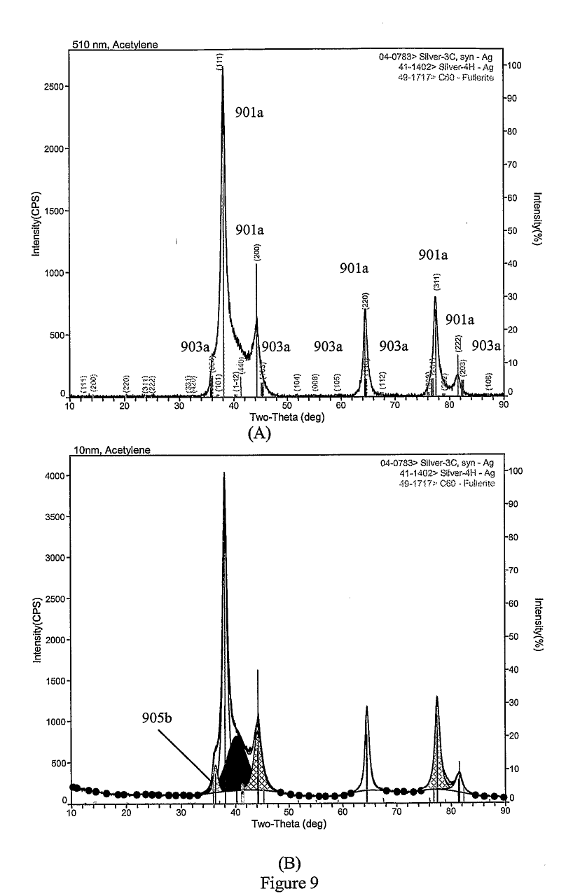

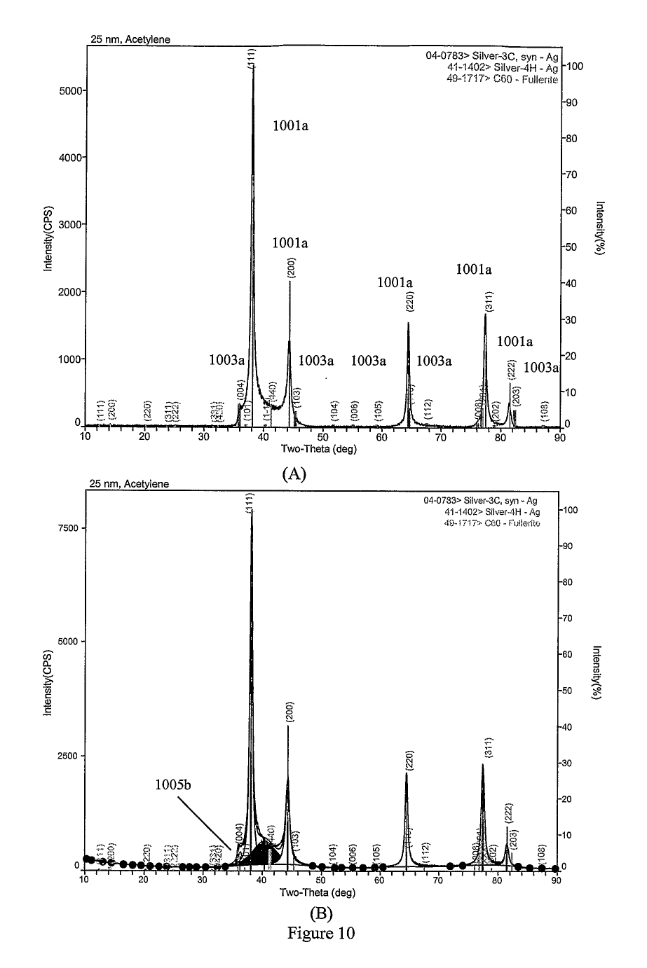

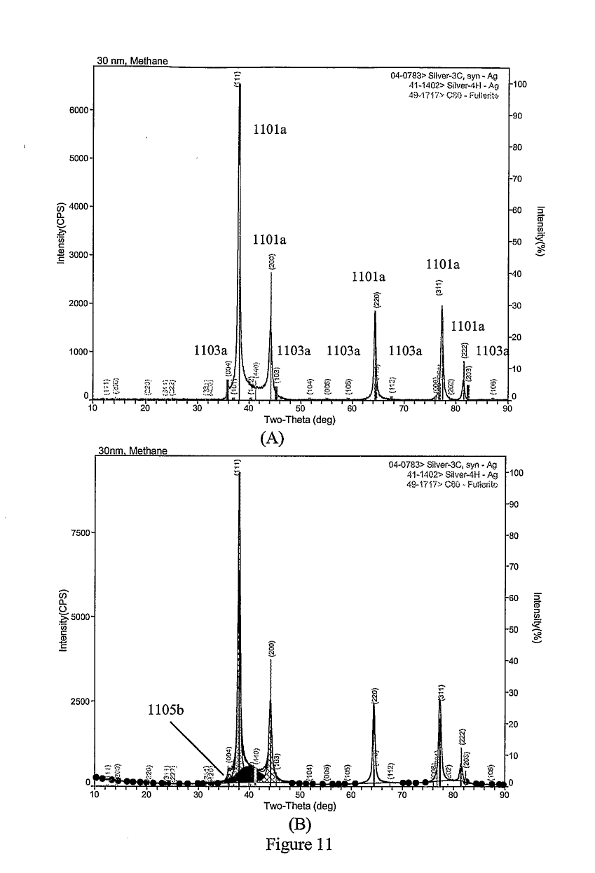

The composition of the produced material was determined from a host of tests including XRD, ICP and LECO. X-ray diffraction tests were performed to determine the material composition and crystal structure. The results are summarized in Table 3. The results confirm that the material is predominantly crystalline silver with a small amount of carbon. The crystallite size of the silver was also estimated from the XRD analysis and shows good correlation with the BET results indicating that the particles are discrete. The XRD analysis showed small amounts of carbon that was interpreted to be Fullerite structures. FIGS. 8-11 show the XRD data for the various materials. In peaks indicated by 801a-1101a (thin lines) in each sample indicate the Face Centered Cubic (FCC) form of silver whereas peaks 803a-1103a (thick lines) indicate the primitive hexagonal form of silver. These results confirm the material is highly crystalline. The peaks indicated by 805b-1105b (solid shaded areas) can be interpreted as fullerite. Later analysis using Scanning Transmission Electron Microscope (STEM), EELS and EDS indicate that this is a mixture of sp2-bonded (graphitic) and sp1-bonded (carbyne) carbon.

TABLE 3

| Gas Con- | | | Crystal | | |
|---|---|---|---|---|---|
| centration | Hydrocarbon | | Size | Metal Crystal | Carbon |
| (ppm) | Gas | BET | (nm) | Structure | Structure |
| Solenoid | None | 25 | 22 | FCC (3C)/ | Fullerite |
| | | | | Hexagonal (4H) | |
| 4400 | Acetylene | 22 | 18 | FCC (3C)/ | Fullerite |
| | | | | Hexagonal (4H) | |
| 44,000 | Acetylene | 10 | 9 | FCC (3C)/ | Fullerite |
| | | | | Hexagonal (4H) | |
| 8800 | Methane | 30 | 22 | FCC (3C)/ | Fullerite |
| | | | | Hexagonal (4H) | |

The metal content was also verified using ICP analysis which shows in excess of 99% silver on a metal basis. This means that of the metal in the sample, 99% is silver. It does not tell the total amount of metal in the sample. From the XRD analysis, the only materials present were carbon and silver. LECO analyses were performed to determine the carbon content in each of the samples. This is shown in the Table 4.

TABLE 4

| Gas | BET | C | N | O |
|---|---|---|---|---|
| None | 77 | 0.0198 | 0.217 | 1.34 |
| 44 ppm C2H2 | 45 | 0.959 | 0.194 | 0.983 |
| 440 ppm C2H2 | 28 | 1.63 | 0.129 | 1.36 |
| 4400 ppm C2H2 | 22 | 3.85 | 0.442 | 1.74 |
| 44000 ppm C2H2 | 9 | 27.9 | 0.480 | 1.93 |
| 8800 ppm CH4 | 30 | 1.38 | 0.209 | 0.618 |

It appears that carbon content increases with gas concentration and the size decreases. Additional testing was performed using GC/MS and FTIR analyses to determine if the carbon is in the form of a hydrocarbon. The GC/MS found only trace amounts of volatile and semi-volatile hydrocarbons. The FTIR results did not show any hydrocarbons confirming the GC/MS results. The FTIR did show that the carbon might be in the form of a cyano derivative. This would indicate that the carbon is partially reacting with the nitrogen.

Analysis of the material was performed using an ultrahigh resolution STEM in conjunction with EELS and Energy Dispersive Spectroscopy (EDS) analysis to quantify the composition of the material. Initial analysis using EELS shows that the particles are silver and that the linear structure is carbon.

Notably, the EELS analysis shows that under certain conditions the carbon has an sp2 and/or sp1 bonding structure. Carbon bonding structure with sp1 is referred to as carbyne. The carbyne structure is elemental carbon in a triply bonded form; rod-like molecule comprised mostly of alkyne (C≡C) groups, more commonly referred to as sp1-bonded chains of carbon atoms. A more generic term is carbyloid which refers to individual types of carbon compounds collectively referred to as carbynes. The two predominate forms of carbyloid are cumulene and polyyne. Cumulene consists of a series of double bonds between carbon atoms (═C═C═C═). Also referred to as allenic carbyne or β-carbyne. Polyyne consists of series of alternating single and triple carbon-carbon bonds (—C≡C—C≡C—). Also referred to as acetylenic carbyne or α-carbyne. The carbyne form of carbon is extremely difficult to produce and has only been produced in laboratories under very specialized conditions. It is generally considered unstable and hence it has been difficult to study.

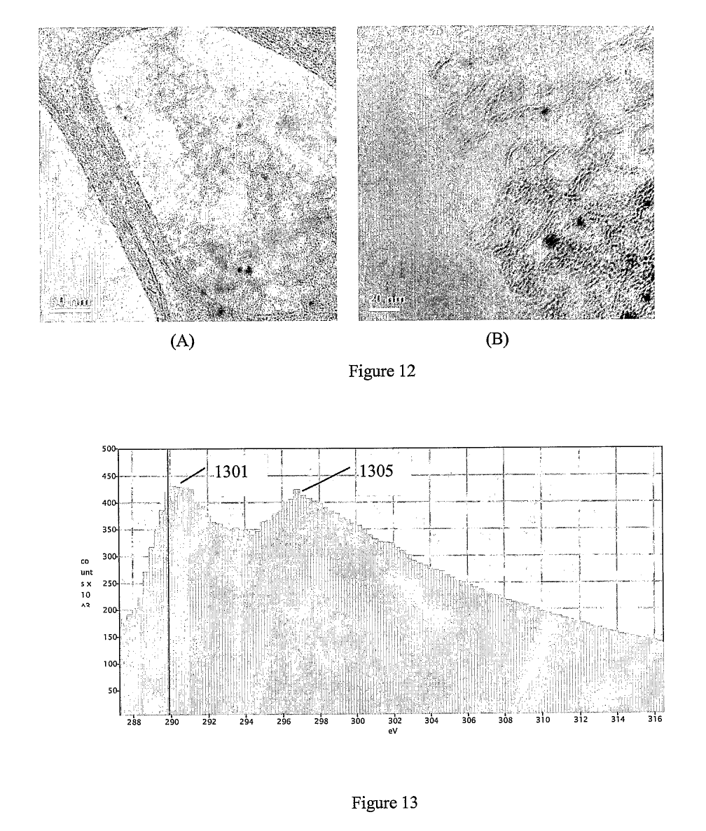

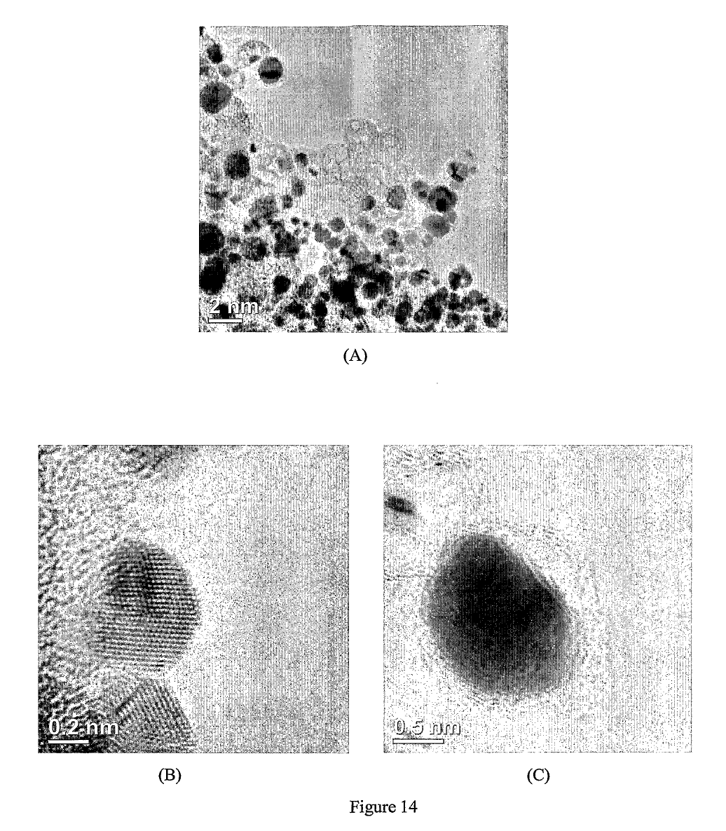

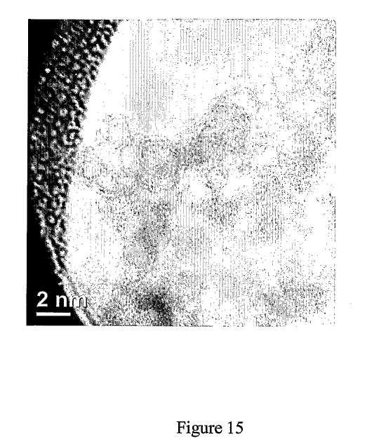

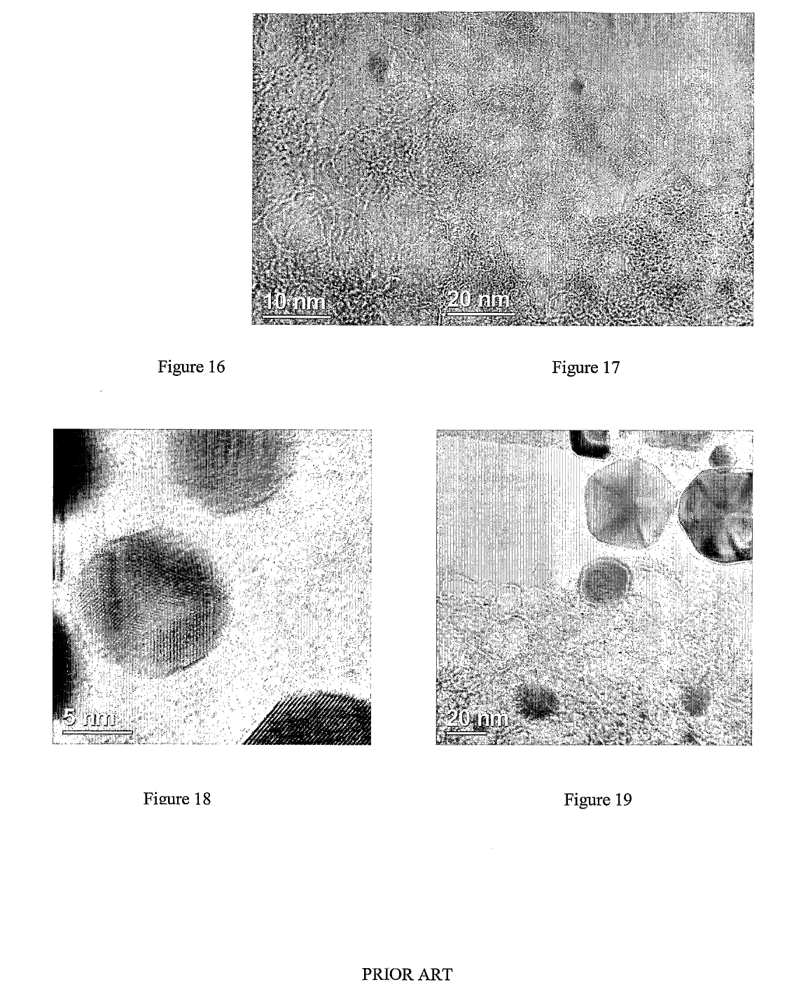

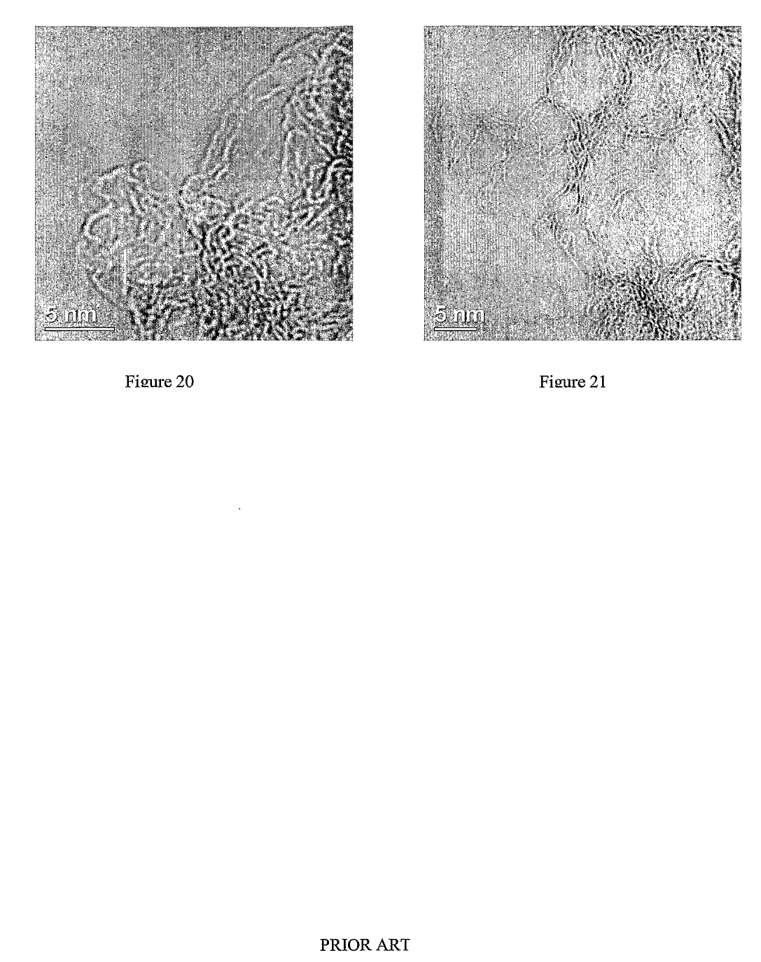

One embodiment of the invention produced by this unique process is a composite of metal particles interspersed within a carbon structure that appears to contain carbyne bonding (sp1). This material can be produced in significant commercial scale quantities, more specifically, silver particles inter-dispersed within a carbon matrix containing carbyne structures. Interestingly, the morphology of the carbon structure appears to change based on the production conditions. One production condition using 4,400-ppm acetylene produces material with a specific surface area of 22 m2/g (BET) that is 97wt. % of silver and 3 wt. % carbon is shown in FIG. 12. It shows discrete silver particles interspersed within a low-density carbon matrix. FIG. 13 shows the EELS data, specifically the Carbon K-edge spectra, of the sample made using 4400 ppm of acetylene. The spectra shows equivalent heights for the □*, 1301, and □*, 1305, peaks indicating the presence of sp1 bonding or carbynes. By increasing the acetylene concentration to 44,000 ppm, the same production conditions yielded a material with a specific surface area of 60 m2/g that is 70 wt. % silver and 30 wt. % carbon. The carbon structure in this material has a different morphology which appear to be “layers” of carbon deposited on the curved surface of the metal nanoparticles as shown in FIG. 14a, 14b and 14c. The dark elements in FIG. 14a show discrete silver particles while the high crystallinity of the silver particles is shown in FIG. 14c as evident by the presence of the lattice planes. For the most part, the silver particles are discrete and are interspersed within the carbon structure. Additionally, the TEM images show that the silver particles are much smaller than the material produced using 4,400 ppm of acetylene. FIG. 15 shows a TEM of the carbynes structures produced using silver and 44,000 ppm of acetylene. When this composite material is exposed to the electron TEM beam, the silver particles are excited and expelled from the carbon matrix. This indicates that the silver particles are not tightly bond to the carbon. For comparison purposes, the material produced with the solenoid process is also shown in FIGS. 16-21. This material appears very different than the material produced with the 44,000 ppm of acetylene.

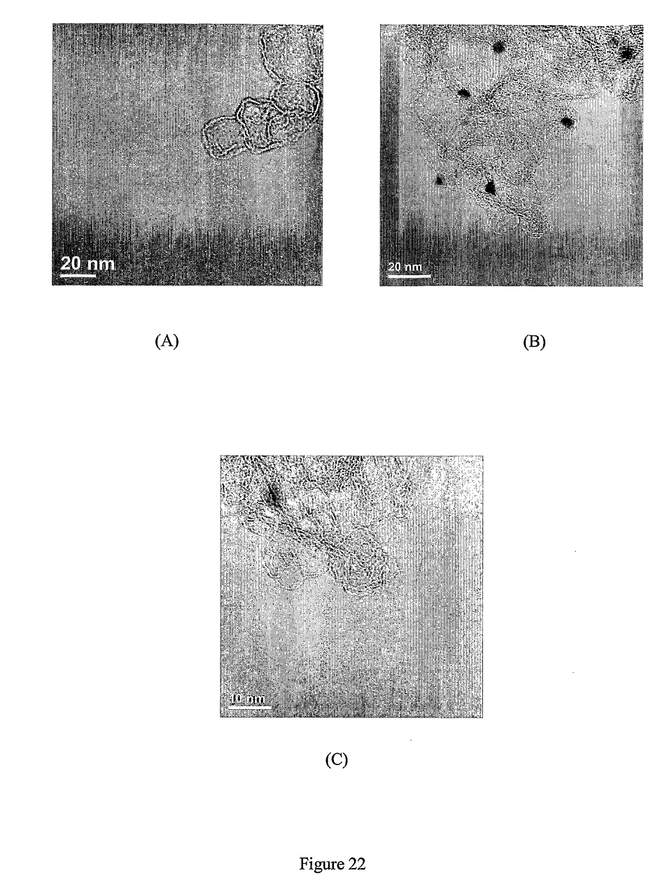

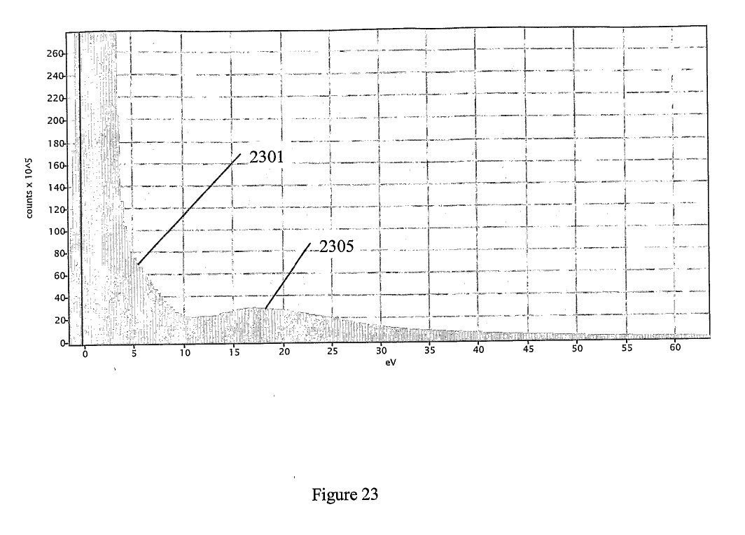

The silver/carbon composite material was also produced using methane at a concentration that has approximately the same amount of carbon as one of the previous conditions. This material gave similar results exhibiting the intertwined layers of carbon with interspersed silver particles as shown in FIGS. 22A-C. The specific surface area was 19 m2/g and with a silver and carbon mass content of 98.5/1.5, respectively. FIG. 23 shows the low loss EELS spectra for the carbon material in the methane silver sample. The peaks 2301 at 4.85 eV and 2305 at 19.5 eV indicate the presence of carbynes. The silver produced using methane did have one notable difference in that when larger quantities were exposed to air it was pyrophoric. The TEM images were taken from a small sample that was isolated before the material ignited. The material produced with acetylene gas was also pyrophoric but only at concentrations below about 500 ppm. For comparison purposes, the solenoid material was not pyrophoric.

While the above example demonstrates the capability of the new invention with silver, it can also be applied to other materials. The following examples show several different materials that were produced using silver, copper, iron, graphite, gold and combinations thereof for the metal precursor. For the most part acetylene gas at various concentrations was used as the hydrocarbon gas. Other hydrogen carbon gases could be used. The following describes the unique materials that were produced.

### EXAMPLE 2

### Copper/Carbon Composite Using Acetylene

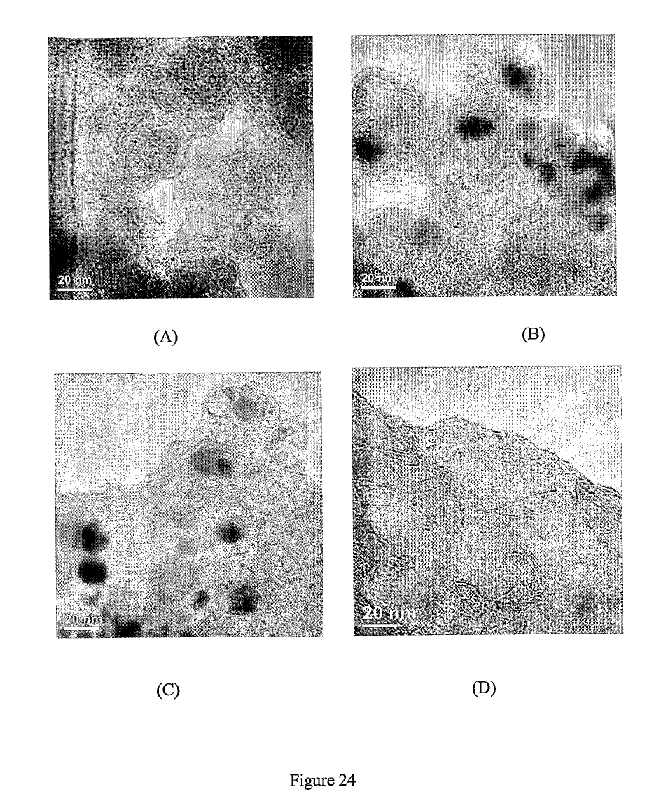

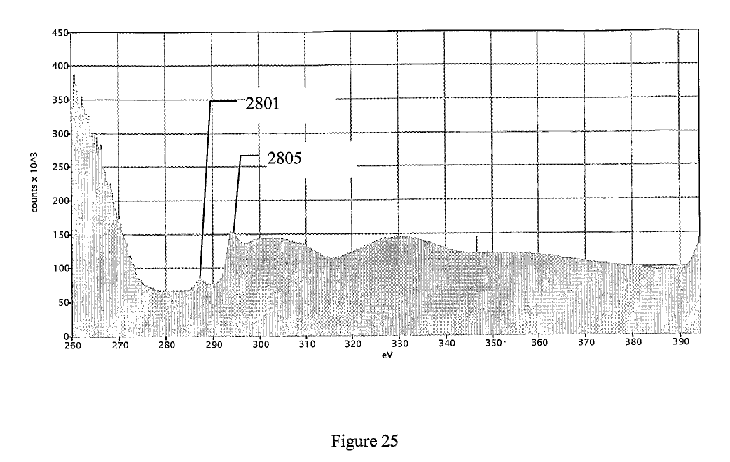

The same production conditions were used to make a copper and carbon composite material. At an acetylene gas concentration of 44,000 ppm, a material with a specific surface area of 44 m2/g and 20 wt % copper and 80 wt % carbon was produced as shown in FIGS. 24A-D. The EELS K-edge spectra is shown in FIG. 25. The high □* peak, 2505, relative to the □* peak, 2501, indicates the material contains a high presence of sp3 carbon (diamond like carbon or fullerene) structures. The □* peak, 2501, also indicates that there is some sp2 carbon or graphitic carbon.

When nanocopper is produced in an inert atmosphere, it will readily oxidize and turn from black to a brownish green when exposed to even small amounts of oxygen. This has been confirmed by XRD analysis. The current nanocopper does not exhibit this feature and XRD analysis confirms that the copper remains copper when exposed to air.

### EXAMPLE 3

### Iron/Carbon Composite Using Acetylene

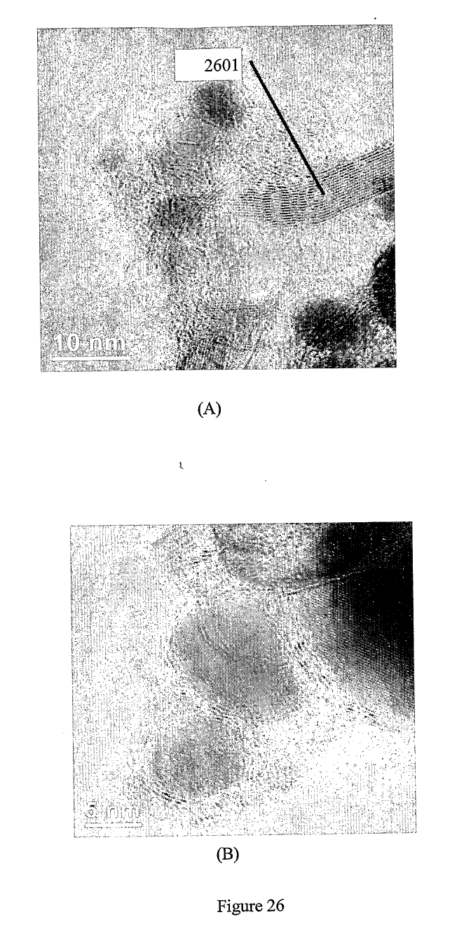

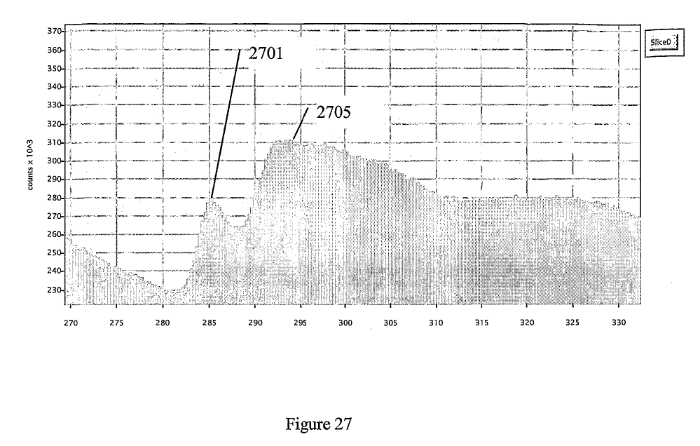

The same production conditions were used to make an iron and carbon composite material. At an acetylene gas concentration of 4,400 ppm, a material with a specific surface area of 65 m2/g was produced as shown in FIGS. 26A-B. TEM images show graphitic structures, 2601, as well as other carbon structures. The EELS K-edge spectra is shown in FIG. 27. The relative heights of □* peak, 2701 and the □* peak, 2705, indicate the particles are interspersed in an sp2-bonded or graphitic carbon matrix. This material is attracted to a magnet and appears to be paramagnetic.

### EXAMPLE 4

### Iron/Silver/Carbon Composite Using Acetylene

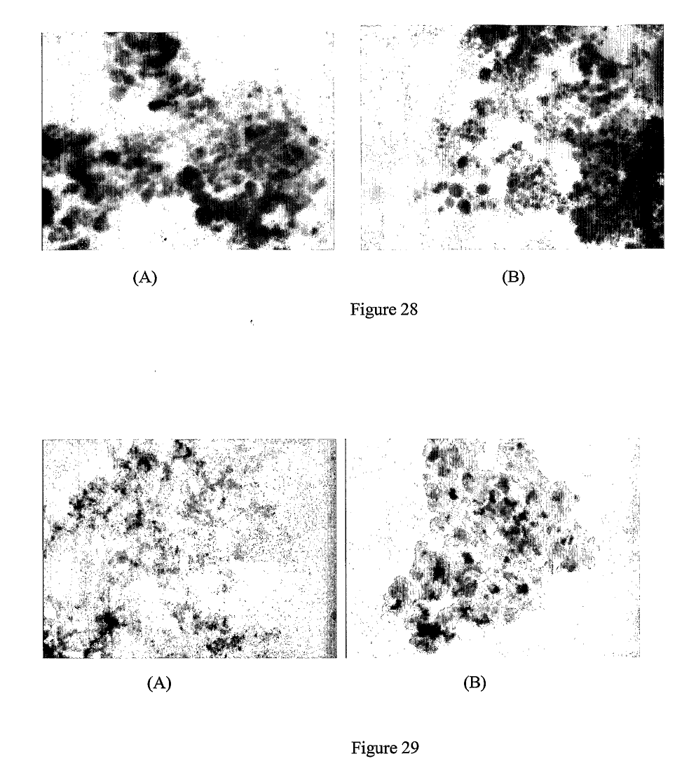

A novel material was synthesized using silver and iron as the precursor metals and acetylene gas as shown in FIGS. 28A-B. The resulting material demonstrated some unique properties, one of which is that the material is magnetic and appears to be paramagnetic. The other unique property is that at certain production conditions the material is pyrophoric when exposed to air.

### EXAMPLE 5

### Graphite Precursor Material

Material was produced using graphite precursor rods and acetylene gas as shown in the TEM images shown in FIGS. 29A-B.

### EXAMPLE 6

### Other Materials

Other materials made with the current invention include a nickel and carbon composite and a nickel/silver and carbon composite. One skilled in the art recognizes that other materials can be fabricated using the gas injection process. Other precious metals such as gold, palladium and platinum can also be used to produce metal/carbon composite materials. These materials are of particular interest because of their catalytic nature, which is similar to silver but generally stronger. This would probably produce more of the carbyne structure. Other metals such as cobalt, aluminum, and other metals can also be used.

For the most part, the material produced with the new process will bum or oxidize when exposed to elevated temperatures, radiation or a flame. It is not clear if this is a pure oxidation reaction of the metal or a chemical reaction involving the carbon matrix.

### Alternate Embodiments

Solid

An alternative embodiment of the current invention involves feeding rods of polycarbonate or other solid material in the vicinity of the arc. In this manner, varying the size, composition, position relative to the arc and the feed rate of the rod can control the amount of material that is removed. Materials such as polycarbonate, thermoplastics such as polyethylene, polypropylene, poly (vinyl chloride), polystyrene, acrylics, nylons and cellulosics, thermoset plastics such as polyamide, polybutadiene, polyether block amide (PEBA), polyetherimide, polyimide, polynrea, polyurethane (PUR), silicone and vinyl ester. Phenolic, melamine and urea formaldehyde could also be used. Fluropolymers such as polytetrafluorethylene (PTFE) and polyvinylidene fluoride (PVDF) may also be used.

It is also possible to control the spatial location where the solid materials are introduced to again control at the point in the quench process where the coating is applied. However, the degree of spatial control would be much more restricted in the case of solids if one depends on the arc plasma to vaporize the material. Supplemental heating can be applied to a solid to induce vaporization at the desired location. An alternative would be to melt, vaporize, or decompose solids external to the reaction chamber and introduce droplets, sprays, or jets in the liquid or gas phase.

Another means of introducing solids into the arc region is the use of a pellet injector. This could either be a simple gravity or mechanically driven injector, or it could be a more sophisticated light gas gun (or similar). It would be possible to use helium as the propellant in a light gas gun to avoid introducing any contaminants.

Liquid

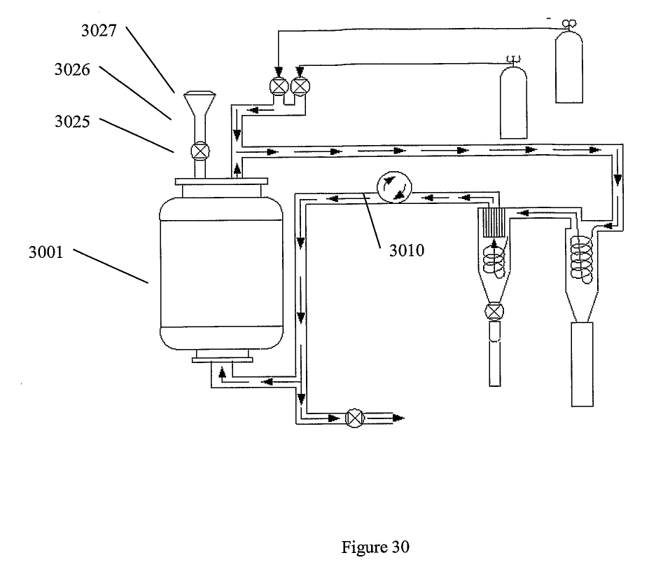

In yet another embodiment, liquid evaporation is used to control the amount of hydrocarbon gas in the system. A test was conducted by the addition of 10 grams and 20 grams, respectively, of heptanethiol directly into the reaction vessel using the apparatus shown in FIG. 30.

In the process, the system is vacuumed and then filled with the inert gas. The ball valve 3025 isolating the liquid port 3026 is closed and the cap from the liquid port is removed. The liquid coating precursor, heptanethiol, is added into the liquid port 3026 and the cap 3027 replaced. The 2-in ball valve 3025 is opened and allows the heptanethiol to enter into the reactor 3001. The blower 3010 is turned on and run for 5-10 minutes to evaporate some of the liquid coating precursor. The reactor 3001 is then operated as normal. By controlling the amount of liquid in the system, the amount of vapor is consequently controlled.

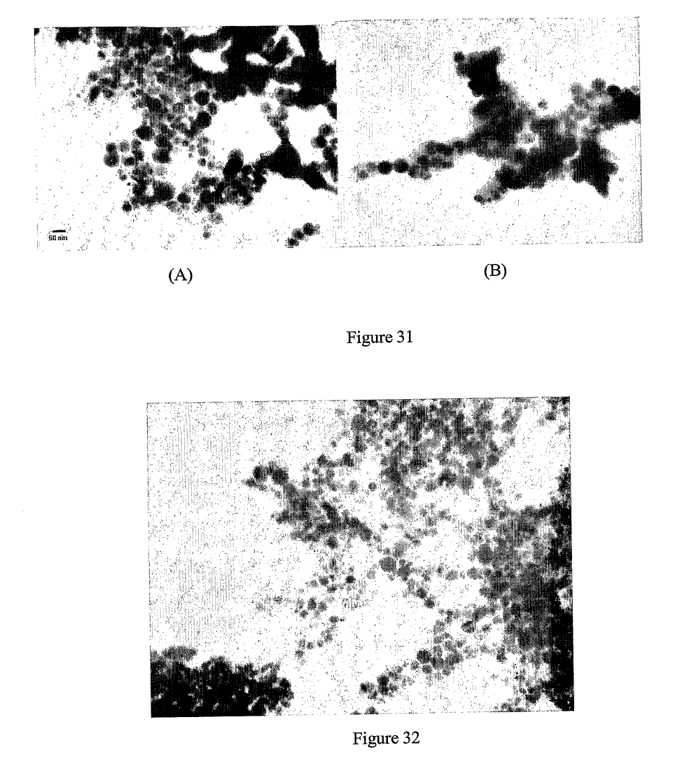

The results of the 10 gm and 20 gm heptanethiol experiments are shown FIGS. 31A-B and FIG. 32, respectively. The materials show that the agglomeration could be reduced, however, there where several aspects that were undesirable. Analysis of the material shows small silver particles coated in carbon or hydrocarbons which then form into agglomerates. Additionally, during collection of the particles, the heptanethiol would condense out and wet the filter resulting in collection problems. Another undesirable aspect was that the amount of vapor could not be as accurately controlled as with the direct gas injection. Lastly, the heptanethiol is difficult to remove from the silver particles. While this system does have some drawbacks, it would be beneficial in a system that uses a wet collection technique.

While the existing test used gas vaporization due to convection and a liquid pool, there are numerous possible secondary material injection systems possible. For example it is possible to use a liquid spray, mist, jet or automated dropper. The choice of system would depend on the molecular weight, vapor pressure, and boiling point of the liquid used. This is only a partial list of the technologies possible for admitting coating liquids.

An alternative type of gas or liquid injection system consists of precisely located pulsed or continuous jets of secondary coating material(s) in the expansion region of the arc synthesized material. This allows controlling the point during the synthesis and quench that the coating material or materials are introduced. This allows partial decoupling of the condensation and coating processes, which allows coating nanoparticles of one material with a material with a higher melting temperature before any agglomeration could occur.

Combination

The gas and liquid phase systems and applications demonstrated to date barely begin to explore the range of possibilities afforded by this technology. This technology enables the production of an entirely new class of nanomaterials with the surface chemical properties of one material and the bulk properties of another material.

The tests have been limited to a narrow range of hydrocarbons and a limited range of concentrations. This technology allows controlling the secondary matrix gas precursor concentration from less than a part per million to 100%. It is possible to introduce more than one material simultaneously by using multiple matrix precursor gases and liquids, as well as electrode materials. By controlling both the arc synthesis parameters and the gas concentration it is possible to independently control the primary particle size, secondary coating thickness, and degree of agglomeration.

It is possible to introduce material into the arc region with a plasma injector. This could include Marshall guns, electrothermal injectors, and other means. In short, coatings could be introduced in solid, liquid, gas, or plasma form resulting in the ability to produce nanomaterials with various coating properties.

Another embodiment includes the simultaneous or staged injection of similar or dissimilar materials by any of the distinct means described above. For example, it is possible to inject a liquid spray of one coating material directly into the arc region while controlling a fixed background concentration of a second coating gas.

Antibacterial Uses

In yet another embodiment the silver nanopowder is used as an antibacterial agent. Silver has long been known to have antimicrobial effects that are a function of the ions released by material. Silver ions can kill bacteria by interfering with respiration (Bragg, P. D. and Rainnie, D. J., “The effect of silver ions on the respiratory chain of Escerhichia coli.,” Can. J. Microbiol. 20, 883-889 (1974)), or by interacting with bacterial DNA (Modak K., and Fox C., “Binding of silver sulfadiazine in the cellular components of Pseudomonas aeruginosa,” Biochem. Pharm. 22: 2392-2404 (1973)). Because of the unique nature of the material, in that the carbon does not appear to be bonded to the surface of the silver as evidence by the particle expulsion in the TEM electron beam, it was speculated that the silver particles would be highly active. Tests were conducted using the 10 nm and 25 nm silver produced using acetylene and 35 nm silver produced using methane. During the testing it was found that the silver had efficacy against several bacteria.

A series of antibacterial efficacy tests were conducted on the new silver using ASTM Standard E2315-03 “Standard Guide for Assessment of Antimicrobial Activity Using a Time-Kill Procedure” protocol to test the new nanopowders against Escherichia coli O157:H7, ATCC 43895 and Staphylococcus aureus, ATCC 6538. The testing incorporated the recommendations described in the “Manual of Clinical Microbiology,” 5th ed., edited by A. B. Balows, ASM, Washington and is directed by the Federal Register, June 1994. Each sample was prepared by mixing the nanopowder in de-ionized water and sonicated using the Misonix Sonicator 3000 for two cycles of 2 minutes at 50% power and 50% duty cycle. The silver was prepared at the higher concentration. The sample at the lower concentration was prepared by dilution of a portion of the higher concentration and then re-sonicated. The results of the test are shown in Table 5 and Table 6.

TABLE 5
Test Results for E. coli O157:H7
Results Expressed as Average Colony Forming Units (CFU) per mL and Percent Reduction
Initial counts: 5.9 × 10⁶

| | Conc. | | | | | |
|---|---|---|---|---|---|---|
| Lot No. | (ppm) | Contact Time | | Replicate | Avg. CFU/mL | Percent Reduction |
| 25 nm Silver, | 100 | 30 | seconds | 1 | >3.0 × 10⁵ | <94.9153 |
| Acetylene | 100 | | | 2 | >3.0 × 10⁵ | <94.9153 |
| | 100 | 1 | Hour | 1 | 1.6 × 10² | 99.9973 |
| | 100 | | | 2 | 1.5 × 10² | 99.9975 |
| 25 nm Silver, | 1000 | 30 | seconds | 1 | >3.0 × 10⁵ | <94.9153 |
| Acetylene | 1000 | | | 2 | >3.0 × 10⁵ | <94.9153 |
| | 1000 | 1 | Hour | 1 | <5.0 × 10⁰ | >99.9999 |
| | 1000 | | | 2 | <5.0 × 10⁰ | >99.9999 |
| 35 nm Silver, | 100 | 30 | seconds | 1 | >3.0 × 10⁵ | <94.9153 |
| Methane | 100 | | | 2 | >3.0 × 10⁵ | <94.9153 |
| | 100 | 1 | Hour | 1 | 5.5 × 10¹ | 99.9991 |
| | 100 | | | 2 | 1.1 × 10² | 99.9981 |
| 35 nm Silver, | 1000 | 30 | seconds | 1 | >3.0 × 10⁵ | <94.9153 |
| Methane | 1000 | | | 2 | >3.0 × 10⁵ | <94.9153 |
| | 1000 | 1 | Hour | 1 | <5.0 × 10⁰ | >99.9999 |
| | 1000 | | | 2 | <5.0 × 10⁰ | >99.9999 |

TABLE 6
Test Results for S. aureus
Results Expressed as Average Colony Forming Units (CFU) per mL and Percent Reduction
Initial counts: 6.2 × 10⁶

| | Conc. | | | | | |
|---|---|---|---|---|---|---|
| Lot No. | (ppm) | Contact Time | | Replicate | Avg. CFU/mL | Percent Reduction |
| 25 nm Silver, | 100 | 30 | seconds | 1 | >3.0 × 10⁵ | <95.1613 |
| Acetylene | 100 | | | 2 | >3.0 × 10⁵ | <95.1613 |
| | 100 | 1 | Hour | 1 | 8.0 × 10¹ | 99.9987 |
| | 100 | | | 2 | 1.5 × 10¹ | 99.9998 |
| 25 nm Silver, | 1000 | 30 | seconds | 1 | >3.0 × 10⁵ | <95.1613 |
| Acetylene | 1000 | | | 2 | >3.0 × 10⁵ | <95.1613 |
| | 1000 | 1 | Hour | 1 | <5.0 × 10⁰ | >99.9999 |
| | 1000 | | | 2 | <5.0 × 10⁰ | >99.9999 |
| 35 nm Silver, | 100 | 30 | seconds | 1 | >3.0 × 10⁵ | <95.1613 |
| Methane | 100 | | | 2 | >3.0 × 10⁵ | <95.1613 |
| | 100 | 1 | Hour | 1 | 3.5 × 10¹ | 99.9994 |
| | 100 | | | 2 | 2.0 × 10¹ | 99.9997 |
| 35 nm Silver, | 1000 | 30 | seconds | 1 | >3.0 × 10⁵ | <95.1613 |
| Methane | 1000 | | | 2 | >3.0 × 10⁵ | <95.1613 |
| | 1000 | 1 | Hour | 1 | <5.0 × 10⁰ | >99.9999 |
| | 1000 | | | 2 | <5.0 × 10⁰ | >99.9999 |

The results from these tests show that both silver nanopowders had an immediate kill of the bacteria and at one hour most had a complete kill. In industry, antibacterial static agents are considered to be materials which prevent growth. These materials typically have at least a Log 0 reduction. Materials are generally considered to have antibacterial properties if there is at least a Log 2 reduction and preferably a Log 3 reduction. Depending on standards, a “complete kill” is defined as between at least a Log 4 or at least a Log 6 percent reduction. While the current tests were performed for one hour, one skilled in the art will recognize the time sensitivity of these tests. Often, additional kill of the bacteria will occur with longer exposure times to the antibacterial agent.

The two bacteria that were tested were chosen because they represent gram positive, S. aureus, and gram negative, E. coli, forms of bacteria. Traditionally silver ions have been shown to be effective against gram-negative bacteria but have been shown to ineffective against gram-positive bacteria. This effect is attributed to the thicker peptidoglycan wall of the gram-positive bacteria. A unique property of the new silver materials is that they had good efficacy against both gram positive and gram-negative bacteria.

In a separate test, the 10 nm silver produced using acetylene gas was tested against E. coli. Concentrations of 100 ppm and 500 ppm were tested and results measured after 5 hours of inoculation. Both concentrations showed 9-log percent reduction in CFU's.

The antibacterial efficacy of the new material can be exploited in many applications. Some areas and products that the new material can be used to give antibacterial properties are wound dressing such as topical wound dressings, creams and ointments. It can be incorporated into various consumer electronics, such as cell phone screens, telephone receivers and keyboards; athletic gear such as shoes, clothing, underwear, protection pads, sweat bands, handle grips, tent surfaces or any other area where there is high moisture exposure such as sweating or water exposure; personal hygiene products such as soaps, deodorant, feminine hygiene pads and personal wipes; dental products such as toothbrushes and dental floss; water filters; humidifiers and wipes to give the item antibacterial properties. It can also be incorporated into various coatings and textiles such architectural epoxies and paints, wood decking and preservation products, awnings, roof covers and pool covers. It can also be used as a biocide for paints, cleaning supplies, pulp and paper, plastics and food products.

The high-current pulsed arc discharge with adjustable feedback-controlled concentration of coating precursor mixture has significant and far-reaching advantages over the competing processes. Either the arc discharge plasma pyrolyses the background gas or liquid vapor, or the hot particles or droplets of the primary material produced during condensation accomplish the same end. This secondary material gas then co-condenses with the primary material. Depending on the relative melting point of the materials, the point at which the secondary material is pyrolysed, and other factors, one or the other material will primarily or entirely reside on the surface of particles composed of the alternate material. By varying the gas concentration and composition, the level of agglomeration and coating thickness can be controlled.

When compared with the previously cited microarc discharge, the current invention delivers roughly 5,000 times more energy to the arc. This allows the current invention to produce orders of magnitude more material per unit time. The higher energy also produces a much hotter plasma with a more rapid quench which enables the synthesis of a much wider range of nanomaterials with superior properties. The new process results in co-condensed particles rather than forming a compound (e.g. metal carbide). It also allows precise control of gas concentration as well as gas mixing.

Comparatively, the microarc experiments were performed with 1 atmosphere of pure methane, whereas the current invention controls the concentration to as low as 1 ppm in an inert gas background. The new nanoparticle coating process produces particles which are far less agglomerated than those produced with the microarc process and are much more crystalline and hence stable.

Compared to the flame synthesis nano-encapsulation processes, the much higher temperature and rapid quench of the new process again allows it to work with a wider range of materials. The flame synthesis process is fundamentally a different process than the current invention and is much more restricted in choice of coating materials and doesn't afford nearly the ability to control the size of particles produced or the range of coating thickness.

Another pre-existing competitor to the new process is the use of chemistry methods such as Sol-Gel to coat one nanomaterial with another. In general, these methods are known to produce soft agglomerates and once the nanoparticles are in solution, it is not possible to dry them without forming aggregates and clumps.

Applicant believes the closest process to the invention of the Application is the Solenoid process. The coating process is an artifact of the solenoid protection armor and is restricted to materials which are compatible with this application. This fact constrains the choice of coating materials to a very short list of high strength, plasma tolerant, insulating, arc resistant materials. Additionally, the decomposition of the liner material results in a wide variety of gas species, the composition of the coating precursor cannot be accurately controlled. Control of the coating process is not possible with the solenoid process. Also the solenoid process introduces significant additional cost and complexity to the synthesis process because it requires a sophisticated solenoid magnet and a large pulsed power supply and control system. Additionally, the solenoid liner needs to be replaced on a periodic basis because it is being consumed. This is a time consuming and labor intensive process. This compares to the addition of a very inexpensive automated gas injection system in the present invention. Finally the solenoid presently used with the solenoid system has a fairly restricted lifetime which limits it to relatively low production applications compared to the current invention.

Significant differences exist between the silver of the current invention and that produced using the solenoid process. First the size range of material exceeded the size range of material produced with the solenoid process. The material also had different dispersion characteristic and the amount of carbyne present was larger. Lastly the material when compared to the other nanosilvers appears to have higher efficacy against bacteria, particularly gram-positive bacteria.

The above Examples are included to demonstrate particular embodiments of the present invention. It should be appreciated by those of skill in the art that the methods disclosed in the examples that follow merely represent exemplary embodiments of the present invention. However, those of skill in the art should, in light of the present disclosure, appreciate that many changes can be made in the specific embodiments described and still obtain a like or similar result without departing from the spirit and scope of the present invention.

All patents and publications referenced herein are hereby incorporated by reference. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. A method comprising:
(a) positioning a pair of electrodes spaced apart in a gaseous atmosphere in a reaction chamber, wherein at least one of said pair of electrodes being a first precursor material and wherein a high power, pulsed power supply is electrically connected across said pair of electrodes;
(b) introducing a second precursor material in a controlled amount into the reaction chamber, wherein the second precursor material is different than the first precursor material;
(c) effecting a high powered electrical discharge from said high power, pulsed power supply across said pair of electrodes to produce a nanopowder comprising generally unagglomerated nanoparticles.

2. A synthesizing system for producing nanopowder comprising:
(a) a reaction chamber having a gaseous atmosphere and a pair of spaced apart electrodes, at least one of which is a first precursor material;
(b) a high power, pulsed power supply electrically connected across said pair of electrodes;
(c) a supply of a second precursor material operatively connected to said reaction chamber; and
(d) a second precursor material controller, wherein the second precursor material controller is operatively connected to the supply of second precursor material and controls the amount of second precursor material that enters the reactor chamber, wherein the nanopowder is produced by effecting a high powered electrical discharge from said high power, pulsed power supply across said pair of electrodes and wherein the nanopowder produced comprises generally unagglomerated nanoparticles.

3. A nanopowder comprising unagglomerated nanoparticles, wherein the nanopowder comprises a metal and carbon and wherein the nanoparticles have an average size less than about 20nm.

4. The nanopowder of claim 3, made by the process comprising:
(a) positioning a pair of electrodes spaced apart in a gaseous atmosphere in a reaction chamber, wherein at least one of said pair of electrodes being a first precursor material and wherein a high power, pulsed power supply is electrically connected across said pair of electrodes;
(b) introducing a second precursor material in a controlled amount into the reaction chamber, wherein the second precursor material is different than the first precursor material;
(c) effecting a high powered electrical discharge from said high power, pulsed power supply across said pair of electrodes to produce a nanopowder comprising generally unagglomerated nanoparticles.

5. The method of claim 1 or the nanopowder of claim 4, wherein the second precursor material is introduced into the reaction chamber in gaseous form.

6. The synthesizing system of claim 2, wherein the supply of the second precursor is in gaseous form.

7. The method of claim 1, the synthesizing system of claim 2, or the nanopowder of claim 4, wherein the second precursor material comprises carbon atoms and wherein said nanopowder comprises carbon atoms from the second precursor material.

8. The method of claim 1, the synthesizing system of claim 2, or the nanopowder of claims 3 or 4, wherein the nanoparticles have an average size in the range between about 8 nm and about 45 nm.

9. The method of claim 1 or the synthesizing system of claim 2, wherein the nanoparticles have an average size in the range between about 8 nm and about 25 nm.

10. The method of claim 1, the synthesizing system of claim 2, or the nanopowder of claims 3 or 4, wherein the nanoparticles have an average size in the range between about 8 nm and 15 nm.

11. The method of claim 1 or the nanopowder of claim 5, wherein the second precursor material is introduced into the reaction chamber at a rate of at least about 44 ppm.

12. The method or the nanopowder of claim 5, wherein the second precursor material is introduced into the reaction chamber at a rate of at least about 440 ppm.

13. The method or the nanopowder of claim 5, wherein the second precursor material is introduced into the reaction chamber at a rate of at least about 4,400 ppm.

14. The method or the nanopowder of claim 5, wherein the second precursor material is introduced into the reaction chamber at a rate of at least about 44,000 ppm.

15. The method or the nanopowder of claim 5, wherein the second precursor material is introduced into the reaction chamber at a rate in the range between about 1 ppm and about 500,000 ppm.

16. The method or the nanopowder of claim 5, wherein the second precursor material is introduced into the reaction chamber at a rate in the range between about 50 ppm and about 50,000 ppm.

17. The method or the nanopowder of claim 5 or the synthesizing system of claim 6, wherein the second precursor material is a hydrocarbon.

18. The method, the synthesizing system, or the nanopowder of claim 17, wherein the hydrocarbon is selected from the group consisting of acetylene, methane, and combinations thereof.

19. The method, the synthesizing system, or the nanopowder of claim 17, wherein the hydrocarbon is selected from the group consisting of alkanes, alkenes, alkynes, and combinations thereof.

20. The method of claim 1, the synthesizing system of claim 2, or the nanopowder of claim 4, wherein the second precursor material is selected from the group consisting of silane gas, borane gas, and combinations thereof.

21. The method of claim 1, the synthesizing system of claim 2, or the nanopowder of claim 4, wherein the first precursor material is selected from the group consisting of silver, copper, aluminum, iron, nickel, zirconium, niobium, gold, platinum, cobalt, titanium, zinc, hafnium, tantalum, tungsten, combinations thereof, alloys thereof, and combinations of the metals and alloys thereof.

22. The method of claim 1, the synthesizing system of claim 2, or the nanopowder of claim 4, wherein the first precursor material is selected from the group consisting of silver, copper, aluminum, iron, nickel, combinations thereof, alloys thereof, and combinations of the metals and alloys thereof.

23. The method of claim 1, the synthesizing system of claim 2, or the nanopowder of claim 4, wherein the first precursor material is selected from the group consisting of zirconium, niobium, gold, platinum, cobalt, titanium, zinc, hafnium, tantalum, tungsten, alloys thereof, and combinations of the metals and alloys thereof.

24. The method of claim 1 or the synthesizing system of claim 2, wherein the nanopowder comprises a metal and carbon.

25. The method or synthesizing system of claim 24 or the nanopowder of claim 3 or 4, wherein the carbon comprises carbyne.

26. The method of claim 1 or the nanopowder of claim 4, wherein the second precursor material is introduced into the reaction chamber in liquid form.

27. The synthesizing system of claim 2, wherein the supply of the second precursor is in liquid form.

28. The method or the nanopowder of claim 26 or the synthesizing system of claim 27, wherein the second precursor material comprises a hydrocarbon.

29. The method or the nanopowder of claim 26 or the synthesizing system of claim 27, wherein the second precursor materials comprises heptanethiol.

30. The method or the nanopowder of claim 26, wherein the step of introducing the second precursor materials comprises using an injection system selected from the group consisting of a liquid spray, mist, jet or automated dropper.

31. The synthesizing system of claim 27, wherein the second precursor material controller comprises an injection system selected from the group consisting of a liquid spray, mist, jet or automated dropper.

32. The method of claim 1 or the nanopowder of claim 4, wherein the second precursor material is introduced into the reaction chamber in solid form.

33. The synthesizing system of claim 2, wherein the supply of the second precursor is in solid form.

34. The method or nanopowder of claim 32, wherein the step of introducing the second precursor material comprises feeding rods in the vicinity of the high powered electrical discharge.

35. The method or nanopowder of claim 32, wherein the step of introducing the second precursor material comprises using a pellet injector.

36. The synthesizing system of claim 33, wherein the second precursor material controller comprises a pellet injector.

37. The method or nanopowder of claim 32, wherein the step of introducing the second precursor is comprises using a device selected from the group consisting of a gravity drive injector, a mechanically driven injector, and a light gas gun.

38. The synthesizing system of claim 33, wherein the second precursor material controller comprises a device selected from the group consisting of a gravity drive injector, a mechanically driven injector, and a light gas gun.

39. The method or nanopowder of claim 32 or the synthesizing system of claim 33, wherein the second precursor material is a material selected from the group consisting of polycarbonates, thermoplastics, thermoset plastics, phenolic formaldehydes, melamine formaldehydes, urea formaldehydes, fluropolymers, and combinations thereof.

40. The method or nanopowder of claim 32 or the synthesizing system of claim 33, wherein the second precursor material is a thermoplastic selected from the group consisting of polyethylene, polypropylene, poly (vinyl chloride), polystyrene, acrylics, nylons, cellulosics, and combinations thereof.

41. The method or nanopowder of claim 32 or the synthesizing system of claim 33, wherein the second precursor material is a thermoset plastic selected from the group consisting of polyamide, polybutadiene, polyether block amide (PEBA), polyetherimide, polyimide, polyurea, polyurethane (PUR), silicone, vinyl ester, and combinations thereof.

42. The method or nanopowder of claim 32 or the synthesizing system of claim 33, wherein the second precursor material comprises a fluropolymer selected from the group consisting of polytetrafluorethylene (PTFE), polyvinylidene fluoride (PVDF), and combinations thereof.

43. The method of claim 1 or the nanopowder of claim 4, wherein the second precursor material is introduced into the reaction chamber in plasma form.

44. The synthesizing system of claim 2, wherein the supply of the second precursor is in plasma form.

45. The method or nanopowder of claim 43, wherein the step of introducing the second precursor materials comprises using a plasma injector.

46. The synthesizing system of claim 44, wherein the second precursor material controller comprises a plasma injector.

47. The method or nanopowder of claim 45 or the synthesizing system of claim 46, wherein the plasma injector is selected from the group consisting of Marshall guns, electrothermal injector, and combination thereof.

48. The method of claim 1 or the nanopowder of claim 4, wherein the second precursor material is introduced into the reaction chamber in at least two forms selected from the group consisting of a gaseous form, liquid form, solid form, and plasma form.

49. The method of claim 1 or the nanopowder of claim 4, further comprising introducing a third precursor material in a controlled manner into the reaction chamber, wherein the second precursor material is introduced into the reaction chamber in a first form selected from the group consisting of a gaseous form, liquid form, solid form, and plasma form, and the third precursor is introduced into the reaction chamber in a second form selected from the group consisting of a gaseous form, liquid form, solid form, and plasma form.

50. The synthesizing system of claim 2, further comprising:
(a) a supply of a third precursor material operatively connected to said reaction chamber; and
(b) a third precursor controller, wherein the third precursor controller is operatively connected to the supply of the third precursor material and controls the amount of third precursor materials the enters the reactor chamber, wherein:
(i) the supply of the second precursor material is in a first form selected from the group consisting of a gaseous form, liquid form, solid form, and plasma form, and
(ii) the supply of the third precursor material is in a second form selected from the group consisting of a gaseous form, liquid form, solid form, and plasma form.

51. The method or the nanopowder of claim 49 or the synthesizing system of claim 50, wherein the first form and second form are different forms.

52. The method or the nanopowder of claim 49 or the synthesizing system of claim 50, wherein the second precursor material and the third precursor material are the same precursor material.

53. The method of claim 1, the synthesizing system of claim 2, or the nanopowder of claim 4, wherein the first precursor material comprises at least two metals.

54. The nanopowder of claim 3, wherein the nanopowder comprises a second metal.

55. The method of claim 1, the synthesizing system of claim 2, or the nanopowder of claim 4, wherein the first precursor material comprises silver.

56. The nanopowder of claim 3, wherein the metal comprises silver.

57. The method, the synthesizing system, or the nanopowder of claim 55, wherein the first precursor material further comprises a second metal.

58. The nanopowder of claim 56, wherein the nanopowder comprises a second metal.

59. The method, the synthesizing system, or the nanopowder of claim 55, wherein the first precursor material further comprises iron.

60. The nanopowder of claim 56, wherein the nanopowder comprises iron.

61. The method, the synthesizing system, or the nanopowder of claim 55, wherein the second precursor material comprises carbon atoms and wherein said nanopowder comprises carbon atoms from the second precursor material.

62. The method of claim 55, further comprising using the nanopowder as an antibacterial agent.

63. The method of claim 1 or the synthesizing system of claim 2, wherein the nanoparticles have an average size less than 20 nm.

64. The method or the synthesizing system of claim 55, wherein the nanoparticles have an average size less than 20 nm.

65. The method or synthesizing system of claim 64 or the nanopowder of claims 55, wherein the second precursor material comprises carbon atoms and wherein said nanopowder comprises carbon atoms from the second precursor material.

66. The method of claim 62, wherein the nanopowder has at least about a Log 2 reduction of bacteria.

67. The synthesizing system of claim 55 or the nanopowder of claims 55 or 56, wherein the nanopowder is operable for use as a bacterial killing agent capable of having at least about a Log 2 reduction of bacteria.

68. The method of claim 62, wherein the nanopowder has at least about a Log 3 reduction of bacteria.

69. The synthesizing system of claim 55 or the nanopowder of claims 55 or 56, wherein the nanopowder is operable for use as a bacterial killing agent capable of having at least about a Log 3 reduction of bacteria.

70. The method of claim 62, wherein the nanopowder has at least about a Log 4 reduction of bacteria.

71. The synthesizing system of claim 55 or the nanopowder of claims 55 or 56, wherein the nanopowder is operable for use as a bacterial killing agent capable of having at least about a Log 4 reduction of bacteria.

72. The method of claim 62, wherein the nanopowder has at least about a Log 6 reduction of bacteria.

73. The synthesizing system of claim 55 or the nanopowder of claims 55 or 56, wherein the nanopowder is operable for use as a bacterial killing agent capable of having at least about a Log 6 reduction of bacteria.

74. The method of claim 62, wherein the nanopowder has a complete kill of bacteria in at least about an hour.

75. The synthesizing system of claim 55 or the nanopowder of claims 55 or 56, wherein the nanopowder is operable for use as a bacterial killing agent capable of having a complete kill of bacteria in at least about an hour.

76. The method of claim 62, wherein the nanopowder is used to reduce or eliminate a type of bacteria selected from the group consisting of gram negative, gram positive and both gram negative and gram positive.

77. The synthesizing system of claim 55 or the nanopowder of claims 55 or 56, wherein the nanopowder is operable for use to reduce or eliminate a type of bacteria selected from the group consisting of gram negative, gram positive and both gram negative and gram positive.

78. The method of claim 76 or the synthesizing system or nanopowder of claim 77, wherein the bacteria is selected from the group consisting of Escherichia coli and Staphylococcus aureus.

79. The method of claim 62, wherein the nanopowder is used in a product selected from the group consisting of electronics, athletic gear, soaps, personal hygiene products, dental products, water filters, humidifiers and wipes.

80. The synthesizing system of claim 55 or the nanopowder of claims 55 or 56, wherein the nanopowder is operable for use as an antibacterial agent in a product selected from the group consisting of electronics, athletic gear, soaps, personal hygiene products, dental products, water filters, humidifiers and wipes.

81. The method of claim 62, wherein the nanopowder is incorporated into a coating.

82. The synthesizing system of claim 55 or the nanopowder of claims 55 or 56, wherein the nanopowder is operable for use as an antibacterial agent in a coating.

83. The method of claim 81 or the synthesizing system or nanopowder of claim 82, wherein the coating is architectural epoxies and paints, wood decking and preservation products and textiles.

84. The method of claim 55, wherein the nanopowder is used as a biocide for a product selected from the group consisting of paints, cleaning supplies, pulp and paper, plastics products, and food products.

85. The synthesizing system of claim 55 or the nanopowder of claims 55 or 56, wherein the nanopowder is operable for use as a biocide for a product selected from the group consisting of paints, cleaning supplies, pulp and paper, plastics products, and food products.

86. The method or synthesizing system of claim 55 or the nanopowder of claims 55 or 56, wherein the carbon comprises carbyne.
